(19) United States
(12) Patent Application Publication
(10) Pub. No.: US 2026/0256756 A1
(43) Pub. Date: Sep. 3, 2026

(54) USE OF PIPERIDINE ALKENE COMPOUND IN PREPARATION OF DRUG FOR TREATING CANCER

(71) Applicants: JIANGSU HANSOH PHARMACEUTICAL GROUP CO., LTD., Jiangsu (CN); SHANGHAI HANSOH BIOMEDICAL CO., LTD., Shanghai (CN)

(72) Inventors: Junhao LIU, Shanghai (CN); Danni SUN, Shanghai (CN); Ting Wang, Shanghai (CN); Hanzhu XIAO, Shanghai (CN); Hongying WEI, Shanghai (CN); Xiaoling QIAN, Shanghai (CN)

(73) Assignees: JIANGSU HANSOH PHARMACEUTICAL GROUP CO., LTD., Jiangsu (CN); SHANGHAI HANSOH BIOMEDICAL CO., LTD., Shanghai (CN)

(21) Appl. No.: 19/163,967

(22) PCT Filed: Mar. 13, 2024
(86) PCT No.: PCT/CN2024/081382

§ 371 Date: Sep. 10, 2025

(30) Foreign Application Priority Data
Mar. 13, 2023 (CN) 202310242242.0

(51) Int. Cl. A61K 31/444; A61P 35/00; A61P 35/04

(52) U.S. Cl. CPC A61K 31/444; A61P 35/00; A61P 35/04

(57) ABSTRACT

Provided is the use of a piperidene alkene compound in the preparation of a drug for treating cancer. Specifically, the use of 1′-((7-ethyl-6-carbonyl-5,6-dihydro-1,5-naphthyridin-3-yl)methyl)-N-methyl-1′,2′,3′,6′-tetrahydro-[3,4′-bipyridine]-6-carboxamide or a pharmaceutically acceptable salt thereof in the preparation of a drug for treating cancer. 1′-((7-ethyl-6-carbonyl-5,6-dihydro-1,5-naphthyridin-3-yl)methyl)-N-methyl-1′,2′,3′,6′-tetrahydro-[3,4′-bipyridine]-6-carboxamide or a pharmaceutically acceptable salt thereof has a good inhibitory effect on each cancer cell, and can be used for treating related cancers.

### TECHNICAL FIELD

The present disclosure relates to the field of clinical medicine and the field of pharmaceuticals, in particular to the use of a piperidine alkene compound in the preparation of a drug for treating of cancer.

### BACKGROUND ART

In countries around the world, malignant tumors are a leading cause of death and a major obstacle to extending life expectancy. According to the World Health Organization evaluation in 2019, among 183 evaluable countries globally, malignant tumors ranked as the first or second leading cause of death in people under 70 years old in 112 countries, including both China and the United States.

PARP is an important part of the base excision repair (BER) pathway, which is the main pathway responsible for repairing single-stranded DNA breaks (SSBs), and is capable of repairing the large number of SSBs produced by human cells under normal physiological and/or pathological conditions. PARP inhibitors can impair the ability of the BER pathway to repair SSBs by inhibiting PARP enzyme catalytic activity and PARP trapping, making SSBs unrepairable and generating a large build-up of SSBs, thereby generating a large number of double-strand DNA breaks (DSBs).

Under normal circumstances, DSB repair can compensate for the loss of BER function. DSB repair pathways mainly include HRR and non-homologous end joining (NHEJ) repair, among others. The former has high precision, and the repaired DNA exhibits high fidelity, making it the main DSB repair method; while the latter, though faster, can result in repair errors, leading to genomic instability and consequently potential cell death. Alterations in HRR genes, such as BRCA1 and BRCA2 mutations, may lead to homologous recombination deficiency (HRD), consequently resulting in unrepairable DNA double-strand breaks or reliance on the mismatch-prone NHEJ pathway for repair, ultimately causing cell death. The combination of both (PARP inhibitor and HRD status) is required to increase cell death.

In 2014, the PARP inhibitor olaparib was conditionally approved in the United States, becoming the first PARP inhibitor approved for marketing and the first anti-tumor drug using the concept of synthetic lethality. To date, the U.S. FDA and China's NMPA have approved six conventional PARP inhibitors, totaling 17 indications, with the vast majority of approved indications requiring patients to have HRR gene mutations (such as BRCA1/2 mutations) or be in an HRD status.

1′-((7-ethyl-6-carbonyl-5,6-dihydro-1,5-diazanaphth-3-yl)methyl)-N-methyl-1′,2,3,6′-tetrahydro-[3,4′-bipyridinyl]-6-carboxamide is a PARP1 inhibitor independently developed by JIANGSU HANSOH PHARMACEUTICAL GROUP CO., LTD./SHANGHAI HANSOH BIOMEDICAL CO., LTD., which has a strong inhibitory effect on the PARP1 enzyme.

### SUMMARY OF THE INVENTION

The present disclosure provides the use of 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof in the preparation of a drug for treating cancer, wherein the cancer is selected from solid tumors.

In certain embodiments of the present disclosure, the solid tumor is an advanced solid tumor.

In certain embodiments of the present disclosure, the solid tumor is a solid tumor that has failed existing standard treatments or is intolerant to existing standard treatments or has no available standard treatments.

In certain embodiments of the present disclosure, the solid tumor is a solid tumor with HRR gene mutation or an HRD-positive solid tumor.

In certain embodiments of the present disclosure, the solid tumor is a solid tumor having one or more of g/sBRCA1/2 mutation, PALB2 mutation, RAD51C mutation, and RAD51D mutation.

In certain embodiments of the present disclosure, the solid tumor is selected from ovarian cancer, breast cancer, pancreatic cancer, prostate cancer, or colorectal cancer; the ovarian cancer is preferably recurrent ovarian cancer; the prostate cancer is preferably metastatic castration-resistant prostate cancer.

In certain embodiments of the present disclosure, the solid tumor is selected from HRD-positive recurrent ovarian cancer, target-positive HER2− breast cancer, target-positive pancreatic cancer, target-positive prostate cancer, target-positive colorectal cancer, wherein the target-positive is one or more of pathogenic and potentially pathogenic g/sBRCA1/2 mutation, PALB2 mutation, RAD51C mutation, and RAD51D mutation.

In certain embodiments of the present disclosure, the HRD-positive recurrent ovarian cancer is selected from recurrent ovarian cancer with g/sBRCA1/2 mutation, recurrent ovarian cancer with PALB2 mutation, recurrent ovarian cancer with RAD51C mutation, and recurrent ovarian cancer with RAD51D mutation.

In certain embodiments of the present disclosure, the HRD-positive recurrent ovarian cancer is selected from recurrent ovarian cancer with pathogenic or likely pathogenic g/sBRCA1/2 mutation or positive genomic instability score detected in a tumor tissue sample.

In certain embodiments of the present disclosure, the target-positive HER2− breast cancer is selected from HER2− breast cancer with g/sBRCA1/2 mutation, HER2− breast cancer with PALB2 mutation, HER2− breast cancer with RAD51C mutation, and HER2− breast cancer with RAD51D mutation.

In certain embodiments of the present disclosure, the target-positive pancreatic cancer is selected from pancreatic cancer with g/sBRCA1/2 mutation, pancreatic cancer with PALB2 mutation, pancreatic cancer with RAD51C mutation, and pancreatic cancer with RAD51D mutation.

In certain embodiments of the present disclosure, the target-positive prostate cancer is selected from prostate cancer with g/sBRCA1/2 mutation, prostate cancer with PALB2 mutation, prostate cancer with RAD51C mutation, and prostate cancer with RAD51D mutation.

In certain embodiments of the present disclosure, the target-positive colorectal cancer is selected from colorectal cancer with g/sBRCA1/2 mutation, colorectal cancer with PALB2 mutation, colorectal cancer with RAD51C mutation, and colorectal cancer with RAD51D mutation.

In certain embodiments of the present disclosure, the breast cancer is triple-negative breast cancer, preferably triple-negative breast cancer with BRCA1 mutation.

In certain embodiments of the present disclosure, the pancreatic cancer is pancreatic cancer with low expression of MRE11A/CHEK2 protein.

In certain embodiments of the present disclosure, the ovarian cancer is ovarian cancer with low expression of ATM protein.

In certain embodiments of the present disclosure, the ovarian cancer is ovarian cancer with BRCA2 mutation.

In certain embodiments of the present disclosure, the pancreatic cancer is pancreatic cancer with BRCA2 mutation.

In certain embodiments of the present disclosure, the prostate cancer is prostate cancer with BRCA2 mutation.

In certain embodiments of the present disclosure, the g/sBRCA1/2 mutation is BRCA1 deletion or BRCA2−/−.

In certain embodiments of the present disclosure, the breast cancer is the MX-1 triple-negative breast cancer.

In certain embodiments of the present disclosure, the pancreatic cancer is the PSN-1 pancreatic cancer.

In certain embodiments of the present disclosure, the ovarian cancer is the CAOV-3 ovarian cancer.

In certain embodiments of the present disclosure, the colorectal cancer is the DLD-1 ovarian cancer.

In certain embodiments of the present disclosure, the pancreatic cancer is the Capan-1 pancreatic cancer.

In certain embodiments of the present disclosure, the colorectal cancer is the DLD-1 BRCA2^{-/-} colorectal cancer.

In certain embodiments of the present disclosure, the prostate cancer is the LNCaP.FGC prostate cancer.

In certain embodiments of the present disclosure, the ovarian cancer is the OV0243 ovarian cancer.

In certain embodiments of the present disclosure, the ovarian cancer is ovarian cancer with BRCA2 gene point mutation.

In certain embodiments of the present disclosure, the pancreatic cancer is pancreatic cancer with BRCA2 gene frameshift mutation.

In certain embodiments of the present disclosure, the colorectal cancer is colorectal cancer with BRCA2 gene deletion mutation.

In certain embodiments of the present disclosure, the prostate cancer is prostate cancer with BRCA2 gene frameshift mutation.

In certain embodiments of the present disclosure, the ovarian cancer is ovarian cancer without pathogenic BRCA1/2 gene mutation.

In certain embodiments of the present disclosure, the solid tumor is selected from HRD-positive recurrent ovarian cancer that has progressed on or is intolerant to at least second-line treatment, target-positive HER2− breast cancer that has progressed on or is intolerant to at least first-line treatment, target-positive pancreatic cancer that has progressed on or is intolerant to at least first-line treatment, target-positive metastatic castration-resistant prostate cancer that has progressed on or is intolerant to at least first-line novel endocrine drugs and at least first-line paclitaxel-containing chemotherapy, and target-positive colorectal cancer that has progressed on or is intolerant to at least second-line treatment, wherein the target-positive is one or more of pathogenic and potentially pathogenic g/sBRCA1/2 mutation, PALB2 mutation, RAD51C mutation, and RAD51D mutation.

In certain embodiments of the present disclosure, the mutation is a germline mutation or a somatic mutation.

In certain embodiments of the present disclosure, the PALB2 mutation is a PALB2 germline mutation or a PALB2 somatic mutation.

In certain embodiments of the present disclosure, the RAD51C mutation is a RAD51C germline mutation or a RAD51C somatic mutation.

In certain embodiments of the present disclosure, the RAD51D mutation is a RAD51D germline mutation or a RAD51D somatic mutation.

In certain embodiments of the present disclosure, the HRD-positive recurrent ovarian cancer that has progressed on or is intolerant to at least second-line treatment is selected from recurrent ovarian cancer with g/sBRCA1/2 mutation that has progressed on or is intolerant to at least second-line treatment, recurrent ovarian cancer with PALB2 mutation that has progressed on or is intolerant to at least second-line treatment, recurrent ovarian cancer with RAD51C mutation that has progressed on or is intolerant to at least second-line treatment, and recurrent ovarian cancer with RAD51D mutation that has progressed on or is intolerant to at least second-line treatment.

In certain embodiments of the present disclosure, the HRD-positive recurrent ovarian cancer that has progressed on or is intolerant to at least second-line treatment is selected from recurrent ovarian cancer with pathogenic or likely pathogenic g/sBRCA1/2 mutation or positive genomic instability score detected in a tumor tissue sample that has progressed on or is intolerant to at least second-line treatment.

In certain embodiments of the present disclosure, the target-positive HER2− breast cancer that has progressed on or is intolerant to at least first-line treatment is selected from HER2− breast cancer with g/sBRCA1/2 mutation that has progressed on or is intolerant to at least first-line treatment, HER2− breast cancer with PALB2 mutation that has progressed on or is intolerant to at least first-line treatment, HER2− breast cancer with RAD51C mutation that has progressed on or is intolerant to at least first-line treatment, and HER2− breast cancer with RAD51D mutation that has progressed on or is intolerant to at least first-line treatment.

In certain embodiments of the present disclosure, the target-positive pancreatic cancer that has progressed on or is intolerant to at least first-line treatment is selected from pancreatic cancer with g/sBRCA1/2 mutation that has progressed on or is intolerant to at least first-line treatment, pancreatic cancer with PALB2 mutation that has progressed on or is intolerant to at least first-line treatment, pancreatic cancer with RAD51C mutation that has progressed on or is intolerant to at least first-line treatment, and pancreatic cancer with RAD51D mutation that has progressed on or is intolerant to at least first-line treatment.

In certain embodiments of the present disclosure, the target-positive metastatic castration-resistant prostate cancer that has progressed on or is intolerant to at least first-line novel endocrine drugs and at least first-line paclitaxel-containing chemotherapy is selected from metastatic castration-resistant prostate cancer with g/sBRCA1/2 mutation that has progressed on or is intolerant to at least first-line novel endocrine drugs and at least first-line paclitaxel-containing chemotherapy, metastatic castration-resistant prostate cancer with PALB2 mutation that has progressed on or is intolerant to at least first-line novel endocrine drugs and at least first-line paclitaxel-containing chemotherapy, metastatic castration-resistant prostate cancer with RAD51C mutation that has progressed on or is intolerant to at least first-line novel endocrine drugs and at least first-line paclitaxel-containing chemotherapy, and metastatic castration-resistant prostate cancer with RAD51D mutation that has progressed on or is intolerant to at least first-line novel endocrine drugs and at least first-line paclitaxel-containing chemotherapy.

In certain embodiments of the present disclosure, the target-positive colorectal cancer that has progressed on or is intolerant to at least second-line treatment is selected from colorectal cancer with g/sBRCA1/2 mutation that has progressed on or is intolerant to at least second-line treatment, colorectal cancer with PALB2 mutation that has progressed on or is intolerant to at least second-line treatment, colorectal cancer with RAD51C mutation that has progressed on or is intolerant to at least second-line treatment, and colorectal cancer with RAD51D mutation that has progressed on or is intolerant to at least second-line treatment.

In certain embodiments of the present disclosure, the pharmaceutically acceptable salt is selected from hydrochloride, sulfate, nitrate, hydrobromide, hydrofluoride, hydroiodide, phosphate, 2,5-dihydroxybenzoate, 1-hydroxy-2-naphthoate, acetate, ethanesulfonate, dichloroacetate, trichloroacetate, acetohydroxamate, adipate, benzenesulfonate, 4-chlorobenzenesulfonate, benzoate, 4-acetamidobenzoate, 4-aminobenzoate, decanoate, hexanoate, octanoate, cinnamate, citrate, cyclohexylaminosulfonate, camphorsulfonate, aspartate, camphorate, gluconate, glucuronate, glutamate, isoascorbate, lactate, malate, mandelate, pyroglutamate, tartrate, dodecyl sulfate, dibenzoyltartrate, ethane-1,2-disulfonate, ethanesulfonate, formate, fumarate, galactonate, gentisate, glutarate, 2-ketoglutarate, glycolate, hippurate, hydroxyethylsulfonate, lactobionate, ascorbate, aspartate, laurate, camphorate, maleate, malonate, methanesulfonate, 1,5-naphthalenedisulfonate, naphthalene-2-sulfonate, nicotinate, oleate, orotate, oxalate, palmitate, pamoate, propionate, salicylate, 4-aminosalicylate, sebacate, stearate, succinate, thiocyanate, undecylenate, trifluoroacetate, benzenesulfonate, p-toluenesulfonate, or L-malate, preferably, hydroxyethylsulfonate, hydrochloride, sulfate, 1,5-naphthalenedisulfonate, methanesulfonate, hydrobromide, ethanesulfonate, phosphate, benzenesulfonate, oxalate, maleate, adipate, citrate, malonate, L-malate, pamoate, p-toluenesulfonate, or fumarate, more preferably, hydrochloride, sulfate, methanesulfonate, or p-toluenesulfonate.

In certain embodiments of the present disclosure, the pharmaceutically acceptable salt is p-toluenesulfonate.

In certain embodiments of the present disclosure, the dose of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is 5 to 300 mg, preferably 10 to 150 mg, more preferably 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg, as well as values between any two of the above values (although not all are listed, they are deemed to be explicitly stated), more preferably 10 mg, 20 mg, 40 mg, 80 mg, or 120 mg.

In certain embodiments of the present disclosure, the administration frequency of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is once daily, twice daily, three times daily, four times daily, once every other day, once a week, twice a week, three times a week, once every other week, three times every two months, four times every two months, five times every two months, twice a month, or once a month.

In certain embodiments of the present disclosure, the administration frequency of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is once daily.

In certain embodiments of the present disclosure, the administration frequency of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is twice daily.

In certain embodiments of the present disclosure, the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is suitable for gastrointestinal administration, injection administration, respiratory administration, or transdermal administration; the gastrointestinal administration is preferably oral administration, sublingual administration, or rectal administration; the injection administration is preferably intravenous injection, intramuscular injection, or subcutaneous injection; oral administration is preferred.

The present disclosure further provides a method for treating cancer as described above, comprising a step of administering to a subject a therapeutically effective dose of 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof.

In certain embodiments of the present disclosure, in the method, the pharmaceutically acceptable salt is as described above.

In certain embodiments of the present disclosure, in the method, the dose of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is 5 to 300 mg, preferably 10 to 150 mg, more preferably 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg, as well as values between any two of the above values (although not all are listed, they are deemed to be explicitly stated), more preferably 10 mg, 20 mg, 40 mg, 80 mg, or 120 mg.

In certain embodiments of the present disclosure, in the method, the administration frequency of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is once daily, twice daily, three times daily, four times daily, once every other day, once a week, twice a week, three times a week, once every other week, three times every two months, four times every two months, five times every two months, twice a month, or once a month.

In certain embodiments of the present disclosure, in the method, the administration frequency of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is once daily.

In certain embodiments of the present disclosure, in the method, the administration frequency of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is twice daily.

In certain embodiments of the present disclosure, in the method, the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is suitable for gastrointestinal administration, injection administration, respiratory administration, or transdermal administration; the gastrointestinal administration is preferably oral administration, sublingual administration, or rectal administration; the injection administration is preferably intravenous injection, intramuscular injection, or subcutaneous injection; oral administration is preferred.

In certain embodiments of the present disclosure, 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide may also represent a polymorph thereof, a solvate thereof, a hydrate thereof, a pharmaceutically acceptable salt thereof, a polymorph of the salt thereof, a hydrate of the salt thereof, and combinations of the foregoing. Specifically, 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide may be free base crystal form A or B, hydrochloride crystal form A, sulfate crystal form A, methanesulfonate crystal form A, p-toluenesulfonate crystal form A, p-toluenesulfonate crystal form B, p-toluenesulfonate crystal form C, p-toluenesulfonate dihydrate crystal form A, benzenesulfonate crystal form A, particularly p-toluenesulfonate dihydrate crystal form A. The free base polymorph can be prepared by the method used in the crystal form study section of the compound in CN 2023113706680. The polymorph of a salt can be prepared by the method used in the salt and crystal form study section of the compound of Example 1 in PCT/CN 2023/125513.

Unless otherwise explained, the terms in the present disclosure have the following meanings:

HRR (homologous recombination repair) refers to homologous recombination repair.

HRD (homologous recombination deficiency) refers to homologous recombination repair deficiency.

HER2− (human epidermal growth factor receptor 2 negative) refers to epidermal growth factor receptor 2 negative.

g/sBRCA1/2 (Germline/somatic breast cancer susceptibility gene 1/2) refers to germline/somatic breast cancer susceptibility gene 1/2.

g/sBRCA1/2m (Germline/somatic breast cancer susceptibility gene 1/2 mutation) refers to germline/somatic breast cancer susceptibility gene 1/2 mutation.

PALB2 (Partner and localizer of breast cancer susceptibility gene 2) refers to partner and localizer of breast cancer susceptibility gene 2.

RAD51C (RAD51 paralog C) refers to RAD51 paralog C.

RAD51D (RADSI paralog D) refers to RAD51 paralog D.

Human pancreatic cancer cell line Capan-1 is a cell line with BRCA2 gene frameshift mutation.

Human colorectal cancer cell line DLD-1 BRCA2^{-/-} is a cell line with BRCA2 gene deletion mutation.

Human breast cancer cell line MX-1 is a cell line with BRCA1 gene frameshift mutation.

Human HuPrime® ovarian cancer OV0243 is a cell line with BRCA2 gene point mutation.

Human prostate cancer cell line LNCaP.FGC is a cell line with BRCA2 gene frameshift mutation.

Human ovarian cancer OV-10-0060 is a cell line without pathogenic BRCA1/2 mutation.

The term “effective dose” refers to an amount of a drug effective to treat a disease or condition in a mammal. In the case of cancer, a therapeutically effective amount of a drug can reduce the number of cancer cells; reduce the size of the tumor; inhibit (i.e., slow to some extent and preferably prevent) the infiltration of cancer cells into surrounding organs; inhibit (i.e., slow to some extent and preferably prevent) tumor metastasis; inhibit tumor growth to some extent; and/or alleviate one or more symptoms associated with the condition to some extent. Depending on the extent to which the drug can prevent the growth of existing cancer cells and/or kill existing cancer cells, it can be cytostatic and/or cytotoxic. For cancer treatment, efficacy in vivo can be measured by evaluating duration of survival, progression-free survival (PFS) duration, response rate (RR), duration of response, and/or quality of life.

### DETAILED DESCRIPTION OF EMBODIMENTS

The present disclosure will be explained in more detail below in conjunction with examples, which are merely illustrative of the technical solutions of the present disclosure and do not limit the spirit and scope of the present disclosure.

Compound 1 in the following test examples refers to 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide, which can be prepared by the method of Example 1 in WO 2022223025.

The experimental instruments required for the test method of the present disclosure are as follows:
Biological safety cabinet (Shanghai Boxun Industry & Commerce Co., Ltd. Medical Equipment Factory, BSC-130011A₂)
CO₂ incubator (Thermo311), centrifuge (Eppendorf 5810R)
Microplate reader (BioTek Synergy H1 or PerkinElmer Envision), pipette (Eppendorf or Rainin)
Carbon dioxide incubator (HERAcell-240i, Thermo Fisher)
Precision balance (SECURA225D-1CN, Sartorius Group, Germany)
Ordinary balance (HZ2002A, Changzhou Tianzhiping Instruments and Equipment Co., Ltd.)
Biological safety cabinet (BSC1300-II-A2, Shinva Medical Instrument Co., Ltd.)
Digital caliper ((0-150) mm/0.01 mm, Mitutoyo, Japan)
Pipette (20-200 μL, 100-1000 μL, Eppendorf)
Refrigerator (HYC-391, Haier), electric pipette-aid (Easypet 3, Eppendorf)
Biological safety cabinet (BSC-130011 A2, Shanghai Boxun Industry & Commerce Co., Ltd. Medical Equipment Factory)
Ultra-clean workbench (CJ-2F, Suzhou Fengshi Experimental Animal Equipment Co., Ltd.)
Thermostat water bath (HWS-12, Shanghai Bluepard Instruments Co., Ltd.), CO₂ incubator (Thermo-311, Thermo)
Centrifuge (Centrifuge 5720R, Eppendorf), magnetic stirrer (08-2G, Chijiu)
Full-automatic cell counter (Countess™ 3, Invitrogen), water purifier (Pacific TII, Thermo)
Digital vernier caliper (CD-6″AX, Mitutoyo, Japan), electronic balance (CPA225D, Sartorius)
Cell culture flask (T25/T75/T225, Corning), electronic balance (BSA323S-CW, Sartorius)
Electronic balance (BSA2202S-CW, Sartorius), ultrasonic cleaner (115F0032, Shanghai Kudos)

### Test Example 1. Study on the In Vitro Proliferation Inhibitory Activity of the Present Disclosure on Various Cancer Cell Lines

1.1 Experimental objective: The objective of this test example is to measure the inhibitory effect of the compound on the proliferation activity of various cancer cell lines.
1.2 Experimental reagents:
BRCA2 Knockout DLD-1 cells, purchased from Creative Biogene; MX-1 cells, purchased from COBIOER BIOSCIENCES CO., LTD.; PSN-1 cells, purchased from COBIOER BIOSCIENCES CO., LTD.; OVCAR-3 cells, purchased from ATCC; CAOV-3 cells, purchased from COBIOER BIOSCIENCES CO., LTD.; Cell Titer-Glo, purchased from Promega (Cat. No. G7573); RPMI 1640, purchased from Gibco (Cat. No. 22400-071); DMEM/F12 purchased from Gibco (Cat. No. 11330-032); DMEM, purchased from Gibco (Cat. No. 11995-065); FBS, purchased from Gibco (Cat. No. 10091148); PBS, purchased from Gibco (Cat. No. 10010023); trypsin, purchased from Gibco (Cat. No. 25200056); Insulin-Transferrin-Se, purchased from Gibco (Cat. No. 41400-045); cell culture plate, purchased from Thermo Company (Cat. No. 165306)
1.3 Experimental method: When separately using RPMI1640 medium, DMEM/F12 medium, and DMEM medium, each containing 10% FBS, to culture BRCA2 Knockout DLD-1, MX-1, PSN-1, OVCAR-3, and CAOV 3 cells to an appropriate cell density, the cells were collected and adjusted with complete medium to an appropriate cell concentration, each cell suspension was plated in 96-well plates, with 90 μL per well, and the plates were placed in a 37° C., 5% CO₂ incubator for overnight adherence. Compound solutions with different concentrations were prepared with DMSO and the medium, and a vehicle control was prepared. The compound solutions were added to the 96-well plates, with 10 μL per well, and the plates were placed in a 37° C., 5% CO₂ incubator for continued culturing for 6-10 days. CellTiter-Glo solution was added and uniformly mixed by shaking. After incubation for 10 minutes in the dark, reading was performed with Synergy H1 or Envision microplate reader.

Experimental data processing method: The luminescence signal value was used to calculate the inhibition rate. Graphpad Prism software was used to fit the concentration and inhibition rate to a nonlinear regression curve, so as to obtain the IC₅₀ value. The results are as shown in Table 1 below:

| | | | IC₅₀ (nM) | | |
|---|---|---|---|---|---|
| Cell name | Cell type | Mutation type | Compound 1 | AZD5305 | Olaparib |
| MX-1 | Triple-negative | BRCA1 mutation | 2.0 ± 0.8 | 4.1 ± 1.6 | 246 ± 104 |
| | breast cancer | | | | |
| PSN-1 | Pancreatic | HRD-positive (low | 2.2 ± 0.3 | 2.1 ± 0.5 | 65 ± 19 |
| | cancer | expression of | | | |
| | | MRE11A/CHEK2 protein) | | | |
| OVCAR-3 | Ovarian cancer | / | 2.8 ± 0.3 | 5.1 ± 1.4 | 319 ± 47 |
| CAOV-3 | Ovarian cancer | HRD-positive (low | 5.3 ± 0.7 | 4.0 ± 0.8 | 151 ± 18 |
| | | expression of ATM | | | |
| | | protein) | | | |
| DLD-1 | Colorectal | BRCA2 deletion | 1.7 ± 0.4 | 2.6 ± 0.6 | 160 ± 66 |
| | cancer | | | | |

Experimental conclusion: Compound 1 of the present disclosure had a strong inhibitory effect on each cell.

### Test Example 2 In Vivo Pharmacodynamic Study of Compound 1 in Subcutaneous Xenograft Tumor Model of Human Pancreatic Cancer Cell Line Capan-1 in Nude Mice

1. Experimental objective: To evaluate the in vivo efficacy of compound 1 in a subcutaneous xenograft tumor model of human pancreatic cancer cell line Capan-1 in nude mice.
2. Reagent
IMDM medium (30-2005, ATCC) and fetal bovine serum (FBS) (35-081-CV, Corning)
DPBS (21-031-CVR, Corning) and Matrigel (354234, Corning)
HPMC (H02J10D78204, Shanghai Yuanye Bio-Technology Co., Ltd.)
Trypsin-EDTA (25200-072, Gibco), penicillin and streptomycin (MA0110, Dalian Meilun Biotechnology Co., Ltd.)
3. Experimental operations and data processing
3.1 Animals:
BALB/c nude mice, 6-8 weeks old, female, purchased from Jiangsu GemPharmatech Co., Ltd.
3.2 Cell culture and cell suspension preparation
a. A vial of Capan-1 cells was taken from a cell bank, the cells were thawed with IMDM medium (IMDM+20% FBS), and the thawed cells were placed in a cell culture flask (with the cell type, date, the name of the culture operator, etc., being marked on the wall of the flask), which were placed in a CO₂ incubator for culturing (wherein the temperature of the incubator was 37° C., and the CO₂ concentration was 5%).
b. The cells were passaged once a week, and after passage, the cells were placed in the CO₂ incubator to continue the culture. This process was repeated until the cell number met the requirements for in vivo efficacy.
c. The cultured cells were collected, counted using a full-automatic cell counter, and resuspended with PBS on the basis of the counting results to prepare a cell suspension (at a density of 5×10⁷/mL). An equal volume of Matrigel was added to the cell suspension, and the resulting mixture was mixed uniformly and then placed in an ice box for later use.
3.3 Cell inoculation
a. Before inoculation, the nude mice were marked with disposable universal ear tags for mice and rats.
b. At the time of inoculation, the cell suspension was mixed uniformly, 0.2-1 mL of the cell suspension was aspirated using a 1 mL syringe with air bubbles expelled, and the syringe was then placed on an ice bag for later use.
c. The nude mouse was secured with the left hand, the right dorsal area near the right shoulder (inoculation site) of the nude mouse was disinfected with a 75% alcohol cotton ball, and inoculation was initiated after 30 seconds.
d. The experimental nude mice were inoculated sequentially (0.2 mL of cell suspension per mouse).
3.4 Tumor measurement, grouping and administration in tumor-bearing mice
a. Tumor was measured on days 4-7 after inoculation depending on the tumor growth, and the tumor size was calculated.

tumor volume (mm³)=length (mm)×width (mm)×width (mm)/2 Tumor volume calculation:
b. According to the body weight of tumor-bearing mice and the size of the tumor, the mice were grouped by random grouping.
c. According to the grouping results, the test drug was started to be administered (administration method: oral administration; administration volume: 10 mL/kg; administration frequency: once/day; administration cycle: 30 days; vehicle: 0.5% HPMC).
d. Tumor measurement and weighing were performed twice a week after starting administration of test drugs.
e. The animals were euthanized at the end of the experiment.
f. Data processing was performed using software such as GraphPad Prism. The tumor inhibitory efficacy of the compound was evaluated by TGI (%) and ΔT/ΔC (%). ΔT/ΔC (%)=(T−T0)/(C−C0)×100, where T and C are the tumor volumes of the treatment group and the vehicle control group at the end of the experiment, respectively, and T0 and C0 are the tumor volumes of the treatment group and the vehicle control group at the beginning of the experiment, respectively. Tumor growth inhibition (TGI) (%)=100−ΔT/ΔC (%). Tumor growth inhibition (TGI) (%)=100−(T−T0)/T0×100 when tumor regression occurs
4. Experimental results and conclusions

TABLE 2
Evaluation of tumor inhibitory efficacy of compound 1 against
human pancreatic cancer Canpan-1 subcutaneous xenograft tumor

| | Tumor volume | | | | |
|---|---|---|---|---|---|
| | (mm³, Mean ± SEM) | | T/C | | p |
| Group | Day 0 | Day 28 | (%) | TGI(%) | value |
| Vehicle | 164 ± 15 | 818 ± 79 | — | — | — |
| Olaparib_100 mg/kg | 164 ± 13 | 724 ± 82 | 86.27 | 14.39 | 0.681 |
| Compound 1 3 mg/kg | 164 ± 13 | 295 ± 42 | 35.11 | 79.99 | <0.001 |
| Compound 1 10 mg/kg | 164 ± 12 | 232 ± 46 | 28.46 | 89.70 | <0.001 |
| Compound 1 30 mg/kg | 164 ± 13 | 207 ± 46 | 24.59 | 93.45 | <0.001 |

Notes:
For p value, compared with the vehicle group, statistical analysis was performed using one-way ANOVA. In addition, the 3 mg/kg, 10 mg/kg, and 30 mg/kg groups of compound 1 were compared with the Olaparib 100 mg/kg group, respectively, and statistical analysis was performed using t-test, with the p values being 0.001, <0.001, and <0.001, respectively.

The results showed that after 29 consecutive days of administration, compound 1 at doses of 3, 10, and 30 mg/kg had TGI of 79.99%, 89.70%, and 93.45%, respectively, exhibiting a significant tumor inhibitory effect, wherein tumor regression was observed in 3 animals (3/8) at 10 mg/kg and 4 animals (4/8) at 30 mg/kg. No body weight abnormalities were observed in each group of mice during the experiment, indicating good tolerability.

### Test Example 3. In Vivo Pharmacodynamic Study of Compound 1 in Subcutaneous Xenograft Tumor Model of Human Colorectal Cancer Cell Line DLD-1 BRCA2^{-/-} in Nude Mice

1. Experimental objective: To evaluate the in vivo efficacy of compound 1 in a subcutaneous xenograft tumor model of human colorectal cancer cell line DLD-1 BRCA2^{-/-} in nude mice.
2. Reagent
RPMI-1640 medium (22400-089, Gibco), fetal bovine serum (FBS) (10099-141C, Gibco)
Phosphate buffer (PBS, 10010-023, Gibco), HPMC (D180HB4005, Shanghai Colorcon Coating Technology Ltd.)
0.25% Trypsin-EDTA (25200-056, Gibco), Pen Strep (P/S) (15140-122, Gibco)
3. Experimental operations and data processing
3.1 Animals: BALB/c nude mice, 6-8 weeks old, female, purchased from the Laboratory Animal Business Department of Shanghai Institute of Planned Parenthood Research.
3.2 Cell culture and cell suspension preparation
a. A vial of DLD-1 BRCA2^{-/-} cells was taken from a cell bank, the cells were thawed with RPMI-1640 medium (RPMI-1640+10% FBS), and the thawed cells were placed in a cell culture flask (with the cell type, date, the name of the culture operator, etc., being marked on the wall of the flask), which were placed in a CO₂ incubator for culturing (wherein the temperature of the incubator was 37° C., and the CO₂ concentration was 5%).
b. The cells were passaged every three days, and after passage, the cells were placed in the CO₂ incubator to continue the culture. This process was repeated until the cell number met the requirements for in vivo efficacy.
c. The cultured cells were collected, counted using a full-automatic cell counter, and resuspended with PBS on the basis of the counting results to prepare a cell suspension (at a density of 5×10⁷/mL), which was then placed in an ice box for later use.
3.3 Cell inoculation
a. Before inoculation, the nude mice were marked with disposable universal ear tags for mice and rats.
b. At the time of inoculation, the cell suspension was mixed uniformly, 0.1-1 mL of the cell suspension was aspirated using a 1 mL syringe with air bubbles expelled, and the syringe was then placed on an ice bag for later use.
c. The nude mouse was secured with the left hand, the right dorsal area near the right shoulder (inoculation site) of the nude mouse was disinfected with a 75% alcohol cotton ball, and inoculation was initiated after 30 seconds.
d. The experimental nude mice were inoculated sequentially (0.1 mL of cell suspension per mouse).
3.4 Tumor measurement, grouping and administration in tumor-bearing mice
a. Tumor was measured on days 10-18 after inoculation depending on the tumor growth, and the tumor size was calculated.

tumor volume (mm³)=length (mm)×width (mm)×width (mm)/2 Tumor volume calculation:
b. According to the body weight of tumor-bearing mice and the size of the tumor, the mice were grouped by random grouping.
c. According to the grouping results, the test drug was started to be administered (administration method: oral administration; administration volume: 10 mL/kg; administration frequency: once/day; administration cycle: 28 days; vehicle: 0.5% HPMC).
d. Tumor measurement and weighing were performed twice a week after starting administration of test drugs.
e. The animals were euthanized at the end of the experiment.
f. Data processing was performed using software such as GraphPad Prism. The tumor inhibitory efficacy of the compound was evaluated by TGI (%) and ΔT/ΔC (%). ΔT/ΔC (%)=(T−T0)/(C−C0)×100, where T and C are the tumor volumes of the treatment group and the vehicle control group at the end of the experiment, respectively, and T0 and C0 are the tumor volumes of the treatment group and the vehicle control group at the beginning of the experiment, respectively. Tumor growth inhibition (TGI) (%)=100−ΔT/ΔC (%). Tumor growth inhibition (TGI) (%)=100−(T−T0)/T0×100 when tumor regression occurs
4. Experimental results and conclusions

TABLE 3
Evaluation of tumor inhibitory efficacy of compound 1
against DLD-1 (BRCA2 deletion) xenograft tumor model

| | Tumor volume | | | | |
|---|---|---|---|---|---|
| | (mm³, Mean ± SEM) | | T/C | TGI | p |
| Group | Day 0 | Day 28 | (%) | (%) | value |
| Vehicle | 154 ± 17 | 1,485 ± 106 | — | — | — |
| AZD5305_0.3 mg/kg | 154 ± 12 | 92 ± 22 | 6.20 | 140.20 | <0.001 |
| Olaparib_100 mg/kg | 154 ± 13 | 942 ± 112 | 63.43 | 40.81 | <0.001 |
| Compound 1_0.3 mg/kg | 154 ± 16 | 31 ± 7 | 2.08 | 179.97 | <0.001 |
| Compound 1_1 mg/kg | 154 ± 15 | 27 ± 6 | 1.81 | 182.50 | <0.001 |
| Compound 1_3 mg/kg | 155 ± 13 | 20 ± 5 | 1.32 | 187.37 | <0.001 |

Notes:
For p value, compared with the vehicle group, statistical analysis was performed using Dunnett's multiple comparisons test in one-way ANOVA. In addition, the compound 1_0.3 mg/kg group was compared with the AZD5305_0.3 mg/kg group, and statistical analysis was performed using t-test, with the p value being 0.0049.

The results showed that after 28 consecutive days of gavage administration, compound 1 at doses of 0.3, 1, and 3 mg/kg had TGI of 179.97%, 182.50%, and 187.37%, respectively, exhibiting a significant tumor inhibitory effect and a good dose-effect relationship; tumor regression was observed in 8 animals (8/8) at three doses of 0.3, 1, and 3 mg/kg. At the same dose (0.3 mg/kg), the tumor inhibitory effect of the compound 1 group was significantly better than that of the positive control AZD5305 group.

### Test Example 4. In Vivo Pharmacodynamic Study of Compound 1 in Subcutaneous Xenograft Tumor Model of Human Breast Cancer Cell Line MX-1 in Nude Mice

1. Experimental objective: To evaluate the in vivo efficacy of the compound in a subcutaneous xenograft tumor model of human breast cancer cell line MX-1 in nude mice.
2. Reagent
RPMI-1640 medium (22400-089, Gibco), fetal bovine serum (FBS) (10099-141C, Gibco)
Phosphate buffer (PBS, 10010-023, Gibco), HPMC (D180HB4005, Shanghai Colorcon Coating Technology Ltd.)
0.25% Trypsin-EDTA (25200-056, Gibco), Pen Strep (P/S) (15140-122, Gibco)
3. Experimental operations and data processing
3.1 Animals: BALB/c nude mice, 8-12 weeks old, female, purchased from the Laboratory Animal Business Department of Shanghai Institute of Planned Parenthood Research.
3.2 Cell culture and cell suspension preparation
a. A vial of MX-1 cells was taken from a cell bank, the cells were thawed with RPMI-1640 medium (RPMI-1640+10% FBS), and the thawed cells were placed in a cell culture flask (with the cell type, date, the name of the culture operator, etc., being marked on the wall of the flask), which were placed in a CO₂ incubator for culturing (wherein the temperature of the incubator was 37° C., and the CO₂ concentration was 5%).
b. The cells were passaged every three days, and after passage, the cells were placed in the CO₂ incubator to continue the culture. This process was repeated until the cell number met the requirements for in vivo efficacy.
c. The cultured cells were collected, counted using a full-automatic cell counter, and resuspended with PBS on the basis of the counting results to prepare a cell suspension (at a density of 3×10⁷/mL), which was then placed in an ice box for later use.
3.3 Cell inoculation
a. Before inoculation, the nude mice were marked with disposable universal ear tags for mice and rats.
b. At the time of inoculation, the cell suspension was mixed uniformly, 0.1-1 mL of the cell suspension was aspirated using a 1 mL syringe with air bubbles expelled, and the syringe was then placed on an ice bag for later use.
c. The nude mouse was secured with the left hand, the right dorsal area near the right shoulder (inoculation site) of the nude mouse was disinfected with a 75% alcohol cotton ball, and inoculation was initiated after 30 seconds.
d. The experimental nude mice were inoculated sequentially (0.1 mL of cell suspension per mouse).
3.4 Tumor measurement, grouping and administration in tumor-bearing mice
a. Tumor was measured on days 10-18 after inoculation depending on the tumor growth, and the tumor size was calculated.

tumor volume (mm³)=length (mm)×width (mm)×width (mm)/2 Tumor volume calculation:
b. According to the body weight of tumor-bearing mice and the size of the tumor, the mice were grouped by random grouping.
c. According to the grouping results, the test drug was started to be administered (administration method: oral administration; administration volume: 10 mL/kg; administration frequency: once/day; administration cycle: 21 days; vehicle: 0.5% HPMC).
d. Tumor measurement and weighing were performed twice a week after starting administration of test drugs.
e. The animals were euthanized at the end of the experiment.
f. Data processing was performed using software such as GraphPad Prism. The tumor inhibitory efficacy of the compound was evaluated by TGI (%) and ΔT/ΔC (%). ΔT/ΔC (%)=(T−T0)/(C−C0)×100, where T and C are the tumor volumes of the treatment group and the vehicle control group at the end of the experiment, respectively, and T0 and C0 are the tumor volumes of the treatment group and the vehicle control group at the beginning of the experiment, respectively. Tumor growth inhibition (TGI) (%)=100−ΔT/ΔC (%). Tumor growth inhibition (TGI) (%)=100−(T−T0)/T0×100 when tumor regression occurs
4. Experimental conclusion

TABLE 4
Evaluation of tumor inhibitory efficacy of compound
1 against MX-1 xenograft tumor model (Day 21)

| | Tumor volume | | | | |
|---|---|---|---|---|---|
| | (mm³, Mean ± SEM) | | T/C | TGI | p |
| Group | Day 0 | Day 21 | (%) | (%) | value |
| Vehicle | 122 ± 16 | 1880 ± 200 | — | — | — |
| AZD5305_3 mg/kg | 122 ± 17 | 642 ± 132 | 34.13 | 70.43 | <0.0001 |
| Olaparib_100 mg/kg | 122 ± 14 | 1656 ± 289 | 88.08 | 12.73 | 0.7465 |
| Compound 1_1 mg/kg | 122 ± 15 | 355 ± 55 | 18.86 | 86.76 | <0.0001 |
| Compound 1_3 mg/kg | 122 ± 17 | 293 ± 44 | 15.60 | 90.25 | <0.0001 |
| Compound 1_10 mg/kg | 122 ± 18 | 236 ± 35 | 12.58 | 93.49 | <0.0001 |

Notes:
Compared with the vehicle group, statistical analysis was performed using Dunnett's multiple comparisons test in One-way ANOVA. In addition, the 0.3 mg/kg, 1 mg/kg, 3 mg/kg, and 10 mg/kg groups of compound 1 were compared with the AZD5305_3 mg/kg group, respectively, and statistical analysis was performed using t-test, with the p values being 0.1727, 0.0507, 0.0183, and 0.0069, respectively; the 0.3 mg/kg, 1 mg/kg, 3 mg/kg, and 10 mg/kg groups of compound 1 were compared with the Olaparib_100 mg/kg group, respectively, and statistical analysis was performed using t-test, with the p values being 0.0193, <0.001, <0.001, and <0.001, respectively.

The results showed that after 21 consecutive days of administration, compound 1 at doses of 1, 3, and 10 mg/kg had TGI of 86.76%, 90.25%, and 93.49%, respectively, exhibiting a significant tumor inhibitory effect and a certain dose-effect relationship; tumor regression was observed in 1 animal (1/8) at the 10 mg/kg dose of compound 1. At the same dose (3 mg/kg), the tumor inhibitory effect of compound 1 was significantly better than that of AZD5305. No body weight abnormalities were observed in each group of mice during the experiment, indicating good tolerability.

### Test Example 5. In Vivo Pharmacodynamic Study of Compound 1 in Subcutaneous Xenograft Tumor Model of Human HuPrime® Ovarian Cancer OV0243 in Nude Mice

1. Experimental objective: To evaluate the in vivo efficacy of the compound in a subcutaneous xenograft tumor model of human HuPrime® ovarian cancer OV0243 in nude mice.
2. Reagent
HPMC (2600-5600cp, SIGMA) and sterilized water for injection (Shijiazhuang No. 4 Pharmaceutical Co., Ltd.)
3. Experimental operations and data processing
3.1 Animals: BALB/c nude mice, 6-7 weeks old, female, purchased from Jiangsu GemPharmatech Co., Ltd.
3.2 Animal modeling
Tumor tissues were harvested from HuPrime® ovarian cancer xenograft model OV0243 tumor-bearing mice and cut into tumor fragments with a diameter of 2-3 mm, which were subcutaneously inoculated into the right anterior scapula region of BALB/c nude mice.
3.3 Tumor measurement and grouping in tumor-bearing mice
Prior to drug administration, all animals were weighed, and tumor volumes were measured using a vernier caliper. The tumor volume was calculated as: tumor volume (mm³)=length (mm)×width (mm)×width (mm)/2. According to the body weight of tumor-bearing mice and the size of the tumor, the mice were grouped by random grouping.
3.4 Administration in tumor-bearing mice
According to the grouping results, the test drug was started to be administered (administration method: oral administration; administration volume: 10 mL/kg; administration frequency: once/day; administration cycle: 28 days; vehicle: 0.5% HPMC). Tumor measurement and weighing were performed twice a week after starting administration of test drugs. The animals were euthanized at the end of the experiment.
3.5 Data processing
Data processing was performed using software such as GraphPad Prism. The tumor inhibitory efficacy of the compound was evaluated by TGI (%) and ΔT/ΔC (%). ΔT/ΔC (%)=(T−T0)/(C−C0)×100, where T and C are the tumor volumes of the treatment group and the vehicle control group at the end of the experiment, respectively, and T0 and C0 are the tumor volumes of the treatment group and the vehicle control group at the beginning of the experiment, respectively. Tumor growth inhibition (TGI) (%)=100−ΔT/ΔC (%). Tumor growth inhibition (TGI) (%)=100−(T−T0)/T0×100 when tumor regression occurs
4. Experimental results and conclusions

TABLE 5
Evaluation of tumor inhibitory efficacy of compound 1 against
human ovarian cancer OV0243 xenograft tumor model

| | Tumor volume | Relative | | | |
|---|---|---|---|---|---|
| | (mm³)^{a} | tumor volume | T/C^{b} | TGI | p |
| Group | (Day 27) | (RTV) | (%) | (%) | value^{c} |
| Vehicle | 1247.4 ± 221.29 | 9.70 ± 0.90 | — | — | — |
| Compound 1, 3 mg/kg | 37.10 ± 8.68 | 0.29 ± 0.05 | 2.97 | 170.02 | <0.001 |

Notes:
^{a}Data was expressed as “mean ± standard error”;
^{b}T/C = T_{RTV}/C_{RTV} × 100%;
^{c}Analysis was performed using Dunnett's multiple comparisons test in One-way Anova to determine a significant difference compared with the Vehicle group.

The results showed that after 28 consecutive days of administration, compound 1 at a dose of 3 mg/kg had a TGI of 170.02%, wherein partial tumor regression was observed in all 6 mice, exhibiting a significant tumor inhibitory effect. No significant abnormalities in body weight and behavior were observed in each group of mice during the experiment, indicating good tolerability.

### Test Example 6. In Vivo Pharmacodynamic Study of Compound 1 in Subcutaneous Xenograft Tumor Model of Human Prostate Cancer Cell Line LNCaP.FGC in Nude Mice

1. Experimental objective: To evaluate the in vivo efficacy of the compound in a subcutaneous xenograft tumor model of human prostate cancer cell line LNCaP.FGC in mice.
2. Reagent
RPMI-1640 medium (22400-089, Gibco), fetal bovine serum (FBS) (10099-141C, Gibco)
Phosphate Buffer (PBS, 10010-023, Gibco), HPMC (D180HB4005, Shanghai Colorcon Coating Technology Ltd.),
0.25% Trypsin-EDTA (25200-056, Gibco), Pen Strep (P/S) (15140-122, Gibco),
Matrigel (356234, Corning)
3. Experimental operations and data processing
3.1 Animals: NOD SCID mice, 7-9 weeks, male, purchased from Zhejiang Vital River Laboratory Animal Technology Co., Ltd.
3.2 Cell culture and cell suspension preparation
a. A vial of LNCaP.FGC cells was taken from a cell bank, the cells were thawed with RPMI-1640 medium (RPMI-1640+10% FBS), and the thawed cells were placed in a cell culture flask (with the cell type, date, the name of the culture operator, etc., being marked on the wall of the flask), which were placed in a CO₂ incubator for culturing (wherein the temperature of the incubator was 37° C., and the CO₂ concentration was 5%).
b. The cells were passaged every three days, and after passage, the cells were placed in the CO₂ incubator to continue the culture. This process was repeated until the cell number met the requirements for in vivo efficacy.
c. The cultured cells were collected, counted using a full-automatic cell counter, and resuspended with PBS on the basis of the counting results to prepare a cell suspension (at a density of 5×10⁷/mL), which was then placed in an ice box for later use.
3.3 Cell inoculation
a. Before inoculation, the mice were marked with disposable universal ear tags for mice and rats.
b. At the time of inoculation, the cell suspension was mixed uniformly, 0.2-1 mL of the cell suspension was aspirated using a 1 mL syringe with air bubbles expelled, and the syringe was then placed on an ice bag for later use.
c. The mouse was secured with the left hand, the hair on the right dorsal area near the right shoulder of the mouse was removed, the inoculation site was disinfected with a 75% alcohol cotton ball, and inoculation was initiated after 30 seconds.
d. The experimental mice were inoculated sequentially (0.2 mL of cell suspension per mouse).
3.4 Tumor measurement, grouping and administration in tumor-bearing mice
a. Tumor was measured on days 9-15 after inoculation depending on the tumor growth, and the tumor size was calculated.

tumor volume (mm³)=length (mm)×width (mm)×width (mm)/2 Tumor volume calculation:
b. According to the body weight of tumor-bearing mice and the size of the tumor, the mice were grouped by random grouping.
c. According to the grouping results, the test drug was started to be administered (administration method: oral administration; administration volume: 10 mL/kg; administration frequency: once/day; administration cycle: 21 days; vehicle: 0.5% HPMC).
d. Tumor measurement and weighing were performed twice a week after starting administration of test drugs.
e. The animals were euthanized at the end of the experiment.
f. Data processing was performed using software such as GraphPad Prism. The tumor inhibitory efficacy of the compound was evaluated by TGI (%) and ΔT/ΔC (%). ΔT/ΔC (%)=(T−T0)/(C−C0)×100, where T and C are the tumor volumes of the treatment group and the vehicle control group at the end of the experiment, respectively, and T0 and C0 are the tumor volumes of the treatment group and the vehicle control group at the beginning of the experiment, respectively. Tumor growth inhibition (TGI) (%)=100−ΔT/ΔC (%). Tumor growth inhibition (TGI) (%)=100−(T−T0)/T0×100 when tumor regression occurs
4. Experimental results and conclusions

TABLE 6
Growth inhibitory effect of compound 1 on LNCaP clone FGC model

| | Mean tumor volume | | | | |
|---|---|---|---|---|---|
| | (mm³) ± SEM | | ΔT/ΔC (%) | TGI (%) | p value |
| Group | D 0 | D 21 | D 21 | D 21 | D 21 |
| Solvent | 140 ± 11 | 850 ± 212 | — | — | — |
| Compound 1, 10 mg/kg | 140 ± 15 | 296 ± 42 | 21.96 | 78.04 | 0.0279 |

Note:
p value D 21: the value was obtained by analyzing the tumor volume of each animal in different groups with the solvent group as the control using t-test.

The compound of the present disclosure showed an excellent tumor inhibitory effect in this model experiment. After 21 consecutive days of oral administration, the compound of the example of the present disclosure could significantly inhibit the growth of LNCaP.FGC mouse xenograft tumors with no significant reduction in animal body weight observed.

### Test Example 7. In Vivo Pharmacodynamic Study of Compound 1 in Subcutaneous Xenograft Tumor Model of Human Ovarian Cancer OV-10-0060 in Nude Mice

1. Experimental objective: To evaluate the in vivo efficacy of the compound in a subcutaneous xenograft tumor model of human ovarian cancer OV-10-0060 in nude mice.
2. Reagent
HPMC (A1921068, Shanghai Aladdin Biochemical Technology Co., Ltd.), DMEM medium (11995-065, Gibco)
3. Experimental operations and data processing
3.1 Animals: BALB/c nude mice, 6-8 weeks old, female, purchased from the Laboratory Animal Business Department of Shanghai Institute of Planned Parenthood Research.
3.2 Animal modeling
Tumor tissues were harvested from ovarian cancer xenograft model OV-10-0060 tumor-bearing mice and cut into tumor fragments with a diameter of 2-3 mm, which were subcutaneously inoculated into the right anterior scapula region of BALB/c nude mice.
3.3 Tumor measurement and grouping in tumor-bearing mice
Prior to drug administration, all animals were weighed, and tumor volumes were measured using a vernier caliper. The tumor volume was calculated as: tumor volume (mm³)=length (mm)×width (mm)×width (mm)/2. According to the body weight of tumor-bearing mice and the size of the tumor, the mice were grouped by random grouping.
3.4 Administration in tumor-bearing mice
According to the grouping results, the test drug was started to be administered (administration method: oral administration; administration volume: 10 mL/kg; administration frequency: once/day; administration cycle: 28 days; vehicle: 0.5% HPMC). Tumor measurement and weighing were performed twice a week after starting administration of test drugs. The animals were euthanized at the end of the experiment.
3.5 Data processing
Data processing was performed using software such as GraphPad Prism. The tumor inhibitory efficacy of the compound was evaluated by TGI (%) and ΔT/ΔC (%). ΔT/ΔC (%)=(T−T0)/(C−C0)×100, where T and C are the tumor volumes of the treatment group and the vehicle control group at the end of the experiment, respectively, and T0 and C0 are the tumor volumes of the treatment group and the vehicle control group at the beginning of the experiment, respectively. Tumor growth inhibition (TGI) (%)=100−ΔT/ΔC (%). Tumor growth inhibition (TGI) (%)=100−(T−T0)/T0×100 when tumor regression occurs
4. Experimental results and conclusions

TABLE 7
Evaluation of tumor inhibitory efficacy of compound 1 against
human ovarian cancer OV-10-0060 subcutaneous xenograft tumor

| | Tumor volume | | | | |
|---|---|---|---|---|---|
| | (mm³, Mean ± SEM) | | T/C | TGI | p |
| Group | Day 0 | Day 27 | (%) | (%) | value |
| Vehicle | 124 ± 4 | 2,257 ± 207 | — | — | — |
| AZD5305, 10 mg/kg | 124 ± 4 | 1,378 ± 99 | 61.08 | 41.19 | <0.001 |
| Olaparib, 100 mg/kg | 124 ± 4 | 1,787 ± 158 | 79.24 | 22.00 | 0.038 |
| Compound 1, 1 mg/kg | 124 ± 4 | 1,215 ± 116 | 53.89 | 48.86 | <0.001 |
| Compound 1, 3 mg/kg | 124 ± 4 | 1,110 ± 95 | 49.26 | 53.75 | <0.001 |
| Compound 1, 10 mg/kg | 124 ± 4 | 1,050 ± 105 | 46.61 | 56.55 | <0.001 |
| Compound 1, 30 mg/kg | 124 ± 4 | 1,034 ± 84 | 45.88 | 57.34 | <0.001 |

Notes:
For p value, compared with the vehicle group, statistical analysis was performed using Dunnett in one-way ANOVA. In addition, the compound 1 10 mg/kg group and the compound 1 30 mg/kg group were compared with the AZD5305 10 mg/kg group, respectively, and statistical analysis was performed using t-test, with the p values being 0.032 and 0.031, respectively; the 1 mg/kg, 3 mg/kg, 10 mg/kg, and 30 mg/kg groups of compound 1 were compared with the Olaparib_100 mg/kg group, respectively, and statistical analysis was performed using t-test, with the p values being 0.006, 0.001, <0.001, and <0.001, respectively.

The results showed that compared with the positive control group, the tumor inhibitory effect of compound 1 at doses of 1 to 30 mg/kg was significantly better than that of Olaparib_100 mg/kg; At the doses of 10 to 30 mg/kg, the tumor inhibitory effect of compound 1 was significantly better than that of AZD5305_10 mg/kg, and at the same dose of 10 mg/kg, the TGI of compound 1 was 15.36% higher than that of AZD5305.

### Test Example 8. Evaluation of the Safety, Tolerability, PK Characteristics, and Efficacy of Compound 1 at Different Doses in Subjects with Advanced Solid Tumors with g/sBRCA1/2m, PALB2 Mutation, RAD51C Mutation, or RAD51D Mutation that have Failed Existing Standard Treatments or are Intolerant to Existing Standard Treatments or have No Available Standard Treatments

### 1. Study Objective:

### Phase Ia (Dose Escalation Phase):

1.1. Primary study objective: To evaluate the safety and tolerability of compound 1 in patients with advanced solid tumors
1.2. Secondary study objective:
1) To evaluate other safety indicators of compound 1 in patients with advanced solid tumors
2) To evaluate the PK characteristics of compound 1 in patients with advanced solid tumors
3) To evaluate the efficacy of compound 1 in patients with advanced solid tumors
1.3. Exploratory study objective
1) To evaluate the relationship between HRR gene alteration and the efficacy of compound 1
2) To evaluate the relationship between ctDNA change and the efficacy of compound 1
3) To evaluate the relationship between exposure and clinical response (including anti-tumor activity and clinical safety)

### Phase Ib (Dose Expansion Phase):

1.1. Primary study objective: To evaluate the efficacy of compound 1 in patients with advanced solid tumors
1.2. Secondary study objective:
1) To evaluate other efficacy indicators of compound 1 in patients with advanced solid tumors
2) To evaluate the safety of compound 1 in patients with advanced solid tumors
3) To evaluate the PK characteristics of compound 1 in patients with advanced solid tumors
1.3. Exploratory study objective
1) To evaluate the relationship between HRR gene alteration or HRD status and the efficacy of compound 1
2) To evaluate the relationship between ctDNA change and the efficacy of compound 1
3) To evaluate the relationship between exposure and clinical response (including anti-tumor activity and clinical safety)

### 2. Study Population: Phase Ia (Dose Escalation Phase):

Patients with advanced solid tumors carrying g/sBRCA1/2m or PALB2 mutation or RAD51C mutation or RAD51D mutation that have failed existing standard treatments or are intolerant to existing standard treatments or have no available standard treatments Phase Ib (dose expansion phase):

On the basis of accumulated clinical trial data, translational medicine research data, and field development progress, appropriate target populations will be selected, including six cohorts: patients with HRD-positive recurrent ovarian cancer that has progressed on or is intolerant to at least second-line treatment, patients with target-positive HER2− advanced breast cancer that has progressed on or is intolerant to at least first-line treatment, patients with target-positive advanced pancreatic cancer that has progressed on or is intolerant to at least first-line treatment, patients with target-positive metastatic castration-resistant prostate cancer that has progressed on or is intolerant to at least first-line novel endocrine drugs and at least first-line paclitaxel-containing chemotherapy, patients with target-positive advanced colorectal cancer that has progressed on or is intolerant to at least second-line treatment, and patients with advanced solid tumors with other HRR gene mutations or with HRD-positive advanced solid tumors, the target-positive referring to one or more of pathogenic and potentially pathogenic g/sBRCA1/2 mutation, PALB2 mutation, RAD51C mutation, or RAD51D mutation) Sample size determination:

The sample size for this study was determined on the basis of clinical rather than statistical considerations. The sample requirements for the two-phase trial were to provide sufficient data to meet the respective study objectives for safety and efficacy evaluations, while minimizing subject exposure to the study products and procedures. In phase Ia, Rolling 6 was used for dose escalation with 3-6 subjects per group. Furthermore, dose expansion might be performed, expanding each group to a maximum of 30 subjects, resulting in an estimated enrollment of 18-102 subjects. In phase Ib, on the basis of accumulated clinical trial data, translational medicine research data, and field development progress, appropriate target populations were selected for group expansion. Each target population included 1-3 dose groups, with a planned enrollment of 20-50 subjects per dose group, resulting in an estimated enrollment of about 300 subjects. In summary, the total estimated enrollment for phase 1 study was 318-402 subjects, and the final actual enrollment would depend on trial progress.

### 3. Administration Regimen:

Subjects began to receive once-daily oral administration under fasting conditions on day 1 (C1D1), and every 28 days was a treatment cycle. The administration continued until objective disease progression (except for compassionate use) or until other protocol-specified criteria for treatment termination were met.

### 4. Evaluation of Results

### 4.1. Safety Evaluation

Within 28 days before the first administration (C1D1), all subjects should undergo screening-phase safety examinations. After being evaluated as eligible for enrollment, baseline safety examinations should be performed before the first administration. After enrollment, all subjects continued to undergo safety evaluations every treatment cycle (4 weeks) until cycle 16, followed by safety evaluations every 8 weeks until 28 days after the last administration. The evaluations included physical examinations, vital signs, laboratory examinations, electrocardiograms, etc.

### 4.2. Efficacy Evaluation:

Within 28 days before the first administration/randomization (C1D1), all subjects should undergo baseline tumor evaluations such as contrast-enhanced CT of the chest, contrast-enhanced CT of the abdomen (including the pelvis), contrast-enhanced MRI (preferred)/contrast-enhanced CT of the head, bone scans, and examinations of other sites with metastatic indications. According to the Response Evaluation Criteria in Solid Tumors (RECIST v1.1), tumor imaging evaluation was performed every 8 weeks after C1D1 until the subject experienced objective disease progression or withdraws from the trial. For ovarian cancer subjects, in addition to RECIST v1.1, efficacy was also evaluated in conjunction with the GCIG CA-125 criteria. For prostate cancer subjects, RECIST v1.1 was only used to evaluate the tumor burden in soft tissues, so the lesion was evaluated according to PCWG3, and the efficacy of compound 1 in prostate cancer was comprehensively evaluated in conjunction with PSA testing.

### 4.3. PK Study

During the study, PK blood samples were collected from subjects to evaluate the PK characteristics of compound 1.

### 4.4. Pharmacodynamic Indicator Evaluation

In the study, blood samples were collected from subjects during the screening phase and administration phase (all subjects in phase Ia+some subjects in phase Ib). Peripheral blood was collected, and circulating tumor DNA (ctDNA) was isolated and detected in an attempt to explore the relationship between ctDNA change and the efficacy of compound 1. For the phase Ib study, pharmacodynamic indicator evaluation was only be performed in some subjects who participated in PK blood collection.

### 4.5. Study on the Relationship Between HRR Gene Mutation Status or HRD Status and the Efficacy of Compound 1

In the study, BRCA1/2 mutation, other HRR gene mutations, or HRD status were collected or detected to evaluate the relationship between the corresponding HRR gene mutation or HRD status and the efficacy of compound 1.

### 4.6. Survival Follow-Up (Phase Ib Only)

Survival follow-up was performed on a 12-week schedule starting from the last administration.

### 4.7. Experimental Results

As of Jan. 25, 2024, in phase Ia (dose escalation phase) study, a total of 3 dose groups completed escalation (3 subjects in the 10 mg QD dose group, 5 subjects in the 20 mg QD dose group, and 3 subjects in the 40 mg QD dose group), and 1 subject was enrolled in the 80 mg dose group in the DLT observation phase. There were 10 subjects with ovarian cancer, 1 subject with breast cancer, and 1 subject with cervical cancer. Among 8 evaluable subjects, 2 subjects achieved confirmed partial response (PR), both from the 10 mg QD dose group and had platinum-sensitive ovarian cancer with no prior PARP inhibitor treatment (1 subject with sBRCAm, and 1 subject with gBRCAm). Both patients showed PR at the first tumor evaluation (C3, 28 days per cycle) with tumor shrinkage of 70% to 75%, and the response persisted until C9, with PFS reaching 7.4 months. No other anti-tumor treatments were incorporated during the trial. The above experimental results showed that compound 1 had anti-tumor activity in subjects with target-positive advanced solid tumors, and exhibited efficacy at low dose levels.

1. A method of treating cancer in a subject in need thereof, the method comprising:
administering to the subject 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof;
wherein the cancer is a solid tumors.

2. The method of claim 1, wherein the solid tumor is a solid tumor with HRR gene mutation or an HRD-positive solid tumor.

3. The method of claim 1, wherein the solid tumor is a solid tumor having one or more of g/sBRCA1/2 mutation, PALB2 mutation, RAD51C mutation, and RAD51D mutation.

4. The method of claim 1, wherein the solid tumor is selected from the group consisting of ovarian cancer, breast cancer, pancreatic cancer, prostate cancer, and colorectal cancer.

5. The method of claim 1, wherein the solid tumor is selected from the group consisting of HRD-positive recurrent ovarian cancer, target-positive HER2-breast cancer, target-positive pancreatic cancer, target-positive prostate cancer, and target-positive colorectal cancer, wherein the target-positive is one or more of pathogenic and potentially pathogenic g/sBRCA1/2 mutation, PALB2 mutation, RAD51C mutation, and RAD51D mutation.

6. The method of claim 1, wherein the solid tumor is selected from the group consisting of HRD-positive recurrent ovarian cancer that has progressed on or is intolerant to at least second-line treatment, target-positive HER2-breast cancer that has progressed on or is intolerant to at least first-line treatment, target-positive pancreatic cancer that has progressed on or is intolerant to at least first-line treatment, target-positive metastatic castration-resistant prostate cancer that has progressed on or is intolerant to at least first-line novel endocrine drugs and at least first-line paclitaxel-containing chemotherapy, and target-positive colorectal cancer that has progressed on or is intolerant to at least second-line treatment, wherein the target-positive is one or more of pathogenic and potentially pathogenic g/sBRCA1/2 mutation, PALB2 mutation, RAD51C mutation, and RAD51D mutation.

7. The method of claim 1, wherein the pharmaceutically acceptable salt is selected from the group consisting of hydrochloride, sulfate, nitrate, hydrobromide, hydrofluoride, hydroiodide, phosphate, 2,5-dihydroxybenzoate, 1-hydroxy-2-naphthoate, acetate, ethanesulfonate, dichloroacetate, trichloroacetate, acetohydroxamate, adipate, benzenesulfonate, 4-chlorobenzenesulfonate, benzoate, 4-acetamidobenzoate, 4-aminobenzoate, decanoate, hexanoate, octanoate, cinnamate, citrate, cyclohexylaminosulfonate, camphorsulfonate, aspartate, camphorate, gluconate, glucuronate, glutamate, isoascorbate, lactate, malate, mandelate, pyroglutamate, tartrate, dodecyl sulfate, dibenzoyltartrate, ethane-1,2-disulfonate, ethanesulfonate, formate, fumarate, galactonate, gentisate, glutarate, 2-ketoglutarate, glycolate, hippurate, hydroxyethylsulfonate, lactobionate, ascorbate, laurate, maleate, malonate, methanesulfonate, 1,5-naphthalenedisulfonate, naphthalene-2-sulfonate, nicotinate, oleate, orotate, oxalate, palmitate, pamoate, propionate, salicylate, 4-aminosalicylate, sebacate, stearate, succinate, thiocyanate, undecylenate, trifluoroacetate, p-toluenesulfonate, and L-malate.

8. The method of claim 1, wherein the dose of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is 5 to 300 mg.

9. The method of claim 1, wherein an administration frequency of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is once daily, twice daily, three times daily, four times daily, once every other day, once a week, twice a week, three times a week, once every other week, three times every two months, four times every two months, five times every two months, twice a month, or once a month.

10. The method of claim 1, wherein the administering comprises gastrointestinal administration, injection administration, respiratory administration, or transdermal administration.

11. The method of claim 1, wherein the solid tumor is recurrent ovarian cancer.

12. The method of claim 1, wherein the solid tumor is metastatic castration-resistant prostate cancer.

13. The method of claim 1, wherein the pharmaceutically acceptable salt is selected from the group consisting of hydrochloride, sulfate, methanesulfonate, and p-toluenesulfonate.

14. The method of claim 11, wherein a dose of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is 10 mg, 20 mg, 40 mg, 80 mg, or 120 mg.

15. The method of claim 12, wherein a dose of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is 10 mg, 20 mg, 40 mg, 80 mg, or 120 mg.

16. The method of claim 6, wherein a dose of the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is 10 mg, 20 mg, 40 mg, 80 mg, or 120 mg.

17. The method of claim 10, wherein the gastrointestinal administration comprises oral administration, sublingual administration, or rectal administration; and the injection administration comprises intravenous injection, intramuscular injection, or subcutaneous injection.

18. The method of claim 14, wherein the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is suitable for oral administration.

19. The method of claim 15, wherein the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is suitable for oral administration.

20. The method of claim 16, wherein the 1′-((7-ethyl-6-carbonyl-5,6-dihydro-1,5-diazanaphth-3-yl)methyl)-N-methyl-1′,2′,3′,6′-tetrahydro-[3,4′-bipyridinyl]-6-carboxamide or a pharmaceutically acceptable salt thereof is suitable for oral administration.
